(19) United States
(12) Patent Application Publication
(10) Pub. No.: US 2021/0088529 A1
(43) Pub. Date: Mar. 25, 2021

(54) USE OF QUINALDINE RED AND DERIVATIVES THEREOF IN PREPARATION OF KIT FOR DETECTING AMYLOID FIBRILS

(71) Applicant: Jilin University, Changchun (CN)

(72) Inventors: Zhijun Chen, Changchun (CN); Haojie Wang, Changchun (CN)

(21) Appl. No.: 16/838,880

(22) Filed: Apr. 2, 2020

(30) Foreign Application Priority Data
Sep. 19, 2019 (CN) 201910888834.3

(51) Int. Cl. G01N 33/68; G01N 33/58

(52) U.S. Cl. CPC G01N 33/6839; G01N 33/582; G01N 2333/62; G01N 2333/936; G01N 2333/75

(57) ABSTRACT

The present invention provides use of quinaldine red (QR) and/or a derivative thereof in preparation of a kit for detecting amyloid fibrils. The derivative includes 4-(4-dimethylaminostyryl)quinoline, and the present invention belongs to the technical field of amyloid fibril detection. Compared with traditional amyloid fibril detection probes, the QR and its derivative 4-(4-dimethylaminostyryl)quinoline have higher binding constants for binding with the amyloid fibrils; and good photobleaching performances; and in the present invention, after combined with the amyloid fibrils, both the QR and its derivative 4-(4-dimethylaminostyryl)quinoline have a fluorescence emission wavelength range that is close to a far-infrared band, and thus are more suitable for imaging a pathological tissue of a biological tissue.

### CROSS-REFERENCE TO RELATED APPLICATIONS

This application claims priority to Chinese patent application 201910888834.3, filed Sep. 19, 2019, the disclosure of which is incorporated herein by reference.

### TECHNICAL FIELD

The present invention relates to the technical field of amyloid fibril detection, and in particular to use of quinaldine red (QR) and/or derivatives thereof in preparation of a kit for detecting amyloid fibrils.

### BACKGROUND

Amyloid fibrils are insoluble protein aggregates. Excessive accumulation of amyloid fibrils in an organ or a tissue can lead to pathological symptoms, leading to various diseases such as Alzheimer's disease, Parkinson's disease and Huntington's disease, spongiform encephalopathy, type II diabetes, arrhythmia, rheumatoid arthritis, atherosclerosis, prolactinoma and polyneuropathy. The research of the amyloid fibrillation involves many disciplinary areas such as chemistry, medicine and biology. Understanding the mechanism of molecular details of amyloid protein deposition process has important diagnostic significance and therapeutic significance. Therefore, a probe for detecting the amyloid fibrils has a great value.

At present, the developed amyloid fibril probes generally include: Thioflavin T (ThT), Congo red, curcumin, etc. Although they can recognize the amyloid fibrils, they also have some shortcomings. For example, for ThT (the most commonly used dye for detecting amyloid fibrils), its green fluorescence emission peak and small Stokes peak shift make the fluorescence have partial overlap with other fluorescent components in a cell, and thus it is not very suitable for in vivo detection.

### SUMMARY

An objective of the present invention is to provide use of quinaldine red (QR) and/or a derivative thereof in preparation of a kit for detecting amyloid fibrils.

In order to realize the objective of the present invention, the present invention provides the following technical solutions.

The present invention provides use of QR and/or a derivative thereof in preparation of a kit for detecting an amyloid fibril, wherein the derivative includes 4-(4-dimethylaminostyryl)quinoline.

Preferably, the QR and/or a derivative thereof are used as fluorescent probes in a kit.

Preferably, the amyloid fibrils include bovine insulin fibrils, lysozyme fibrils, fibrinogen, and/or Aβ amyloid fibrils.

Beneficial effect of the present invention: The present invention provides the use of QR and/or a derivative thereof in preparation of a kit for detecting the amyloid fibrils, wherein the derivative includes 4-(4-dimethylaminostyryl)quinoline. Compared with a traditional amyloid fibril detection probe, the QR and/or its derivative 4-(4-dimethylaminostyryl)quinoline have higher binding constants for binding with the amyloid fibril; and good photobleaching performance; and in the present invention, after combined with the amyloid fibrils, the QR and/or its derivative 4-(4-dimethylaminostyryl)quinoline have a fluorescence emission wavelength range that is close to a far-infrared band, and thus is more suitable for imaging pathological regions of a biological tissue.

### BRIEF DESCRIPTION OF THE DRAWINGS

FIG. 1 shows the analysis results of the response of QR to bovine insulin fibrils in Embodiment 1, where panel A shows the fluorescence response of QR to the bovine insulin fibrils and a detection limit thereof; and panel B is a linear interval of QR identification of the bovine insulin fibril (the fluorescence response).

FIG. 2 shows the results of a study on the manner of binding between QR and the bovine insulin in Embodiment 2, which are Raman signals (at an excitation wavelength of 532 nm) of QR (curve 2), the bovine insulin (curve 3), and QR+the bovine insulin (curve 1), respectively.

FIG. 3 shows the analysis results of a performance of the QR in detecting the amyloid fibril in Embodiment 3, where FIG. 3-A is identification of the bovine insulin fibrils by QR, where A1 is QR+the bovine insulin, A2 is QR, and A3 is QR+the bovine insulin fibrils; FIG. 3-B shows the process of bovine insulin fibrillation as detected by the QR; FIG. 3-C is identification of the lysozyme fibril by QR, where A1 is QR+lysozyme, A2 is QR, and A3 is QR+the lysozyme fibril; and FIG. 3-D shows the process of lysozyme fibrillation as detected by QR.

FIG. 4 shows the binding effects of various amyloid probes on the bovine insulin fibrils as determined by ITC in Embodiment 4, where FIG. 4-A shows titration of QR by the bovine insulin fibrils; FIG. 4-B shows titration of ThT by the bovine insulin fibrils; FIG. 4-C shows the titration of Thioflavin S (ThS) by the bovine insulin fibrils; and FIG. 4-D shows titration of nile red (NR) by the bovine insulin fibrils.

FIG. 5 shows QR replacement of a conventional probe that has bound to the bovine insulin fibrils in Embodiment 5: the bovine insulin is incubated in 25 mM NaCl/HCl (pH=1.6) at 65° C. for 240 min. Panel A shows fluorescence intensity curves respectively with the addition of QR (curve 3, excited at 565 nm), ThT (curve 2, excited at 440 nm), ThS (curve 1, excited at 355 nm), and NR (curve 4, excited at 600 nm). Panel B shows the fluorescence intensities of the bovine insulin fibrils excited at 440 nm (curves 1 and 2) and 565 nm (curves 3 and 4) respectively after added with ThT and after added with ThT and then QR. Panel C shows the fluorescence intensities of the bovine insulin fibrils excited at 355 nm (curves 1 and 2) and 565 nm (curves 3 and 4) respectively after added with ThS and after added with ThS and then QR. Panel D shows the fluorescence intensities of the bovine insulin fibrils excited at 600 nm (curves 1 and 2) and 565 nm (curves 3 and 4) respectively after added with NR and after added with NR and then QR.

FIG. 6 shows photobleaching experiments of QR, ThT, ThS, and NR under ultraviolet light (365 nm) irradiation after being mixed with the bovine insulin fibrils, where samples are taken every half hour and respectively determined for fluorescence on a fluorescence spectrometer. After statistical analysis, a fluorescence change graph of fluorescence intensity over an illumination time is plotted.

FIG. 7 shows the identification (fluorescence response) of the bovine insulin fibrils by 4-(4-dimethylaminostyryl)quinoline, where curve 1 is 4-(4-dimethylaminostyryl)quinoline+bovine insulin, curve 2 is 4-(4-dimethylaminostyryl)quinoline, curve 3 is 4-(4-dimethylaminostyryl)quinoline+bovine insulin fibrils, and the excitation wavelength is 565 nm.

### DETAILED DESCRIPTION

The present invention provides use of QR and/or a derivative thereof in preparation of a kit for detecting an amyloid fibril, where the derivative includes 4-(4-dimethylaminostyryl)quinoline.

In the present invention, QR is crimson powder and is dissolved in water; the structural formula of QR is shown in formula I; QR is preferably purchased from Shanghai regal Biology Technology Co, Ltd.; and the 4-(4-dimethylaminostyryl)quinoline is preferably purchased from TCI.

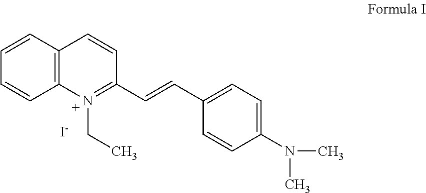

In the present invention, QR and/or a derivative thereof are preferably used as fluorescent probes in a kit; and compared with a traditional amyloid fibril detection probe, both the QR and its derivative 4-(4-dimethylaminostyryl)quinoline (Formula II) have higher binding constants of binding with the amyloid fibrils.

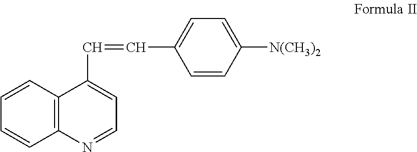 [SMILES: CN(C)c1ccc(C=Cc2ccnc3ccccc23)cc1]

In the present invention, the amyloid fibril preferably includes one or more of a bovine insulin fibril, a lysozyme fibril, an α-synuclein fibril, an Aβ amyloid fibril, a Tau protein fibril, a transthyretin fibril, a serum amyloid protein A fibril, an amylin fibril, a gelsolin fibril, a microglobulin fibril, a prolactin fibril, a prion fibril, a Huntington protein fibril, a calcitonin fibril, an atrial natriuretic peptide fibril, an apolipoprotein A1 fibril, a lactadherin fibril, a transforming growth factor fibril, an immunoglobulin light chain fibril, and the like protein fibrils.

The technical solution provided by the present invention will be described in detail in connection with the following embodiments, but they should not be construed as limiting the claimed scope of the present invention.

### Embodiment 1 Analysis of Response of QR to Bovine Insulin Fibrils

### Reagents

Formulation of a PBS buffer: a mixture of 19 mL of a 0.2 M NaH2PO4 aqueous solution and 81 mL of a 0.2 M Na2HPO4 aqueous solution.

Formulation of a 25 mM NaCl/HCl solution: the pH of the NaCl aqueous solution was adjusted to 1.6 by using HCl, and the concentration of NaCl in the NaCl aqueous solution was 25 mM.

### Method

1) Bovine insulin was added into 1 mL of a NaCl/HCl (25 mM, pH=1.6) solution, where the bovine insulin had a concentration of 0.5 mM, and was incubated at 65° C. for 240 min.

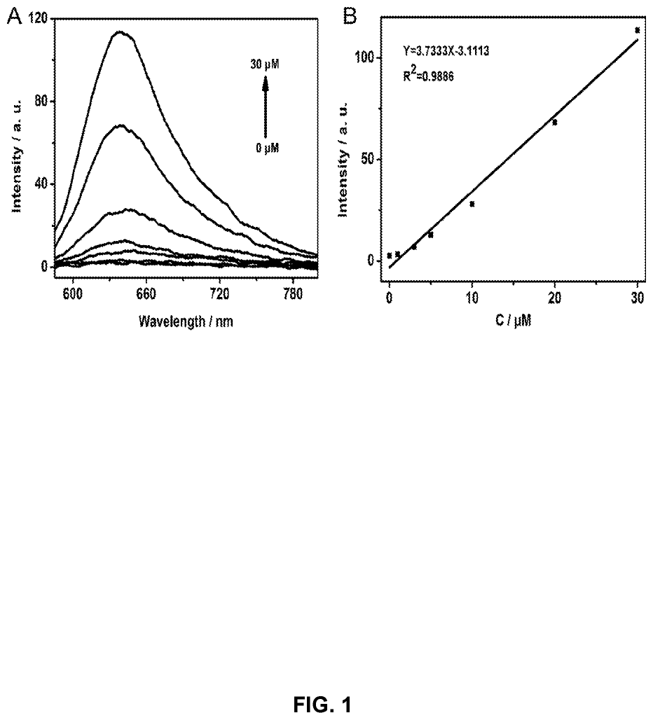

2) The bovine insulin fibrils were diluted with PBS to 0 μm, 1 μm, 3 μm, 5 μm, 10 μm, 20 μm and 30 μm respectively. Then the diluted samples were each added with 0.05 mL of ThT (1 mM) solution, and tested in a fluorescence spectrometer for the fluorescence intensity (λex=440 nm). The fluorescence spectrum showed that the fluorescence intensity increased with the increase of the fibril concentration, and the response of QR to the bovine insulin fibrils is presented in a linear relation in the range of 0-30 μm. The results are shown in FIG. 1, where FIG. 1-A was the detection limit of QR on the bovine insulin fibrils; and FIG. 1-B was a linear interval for QR identification of the bovine insulin fibrils.

### Embodiment 2

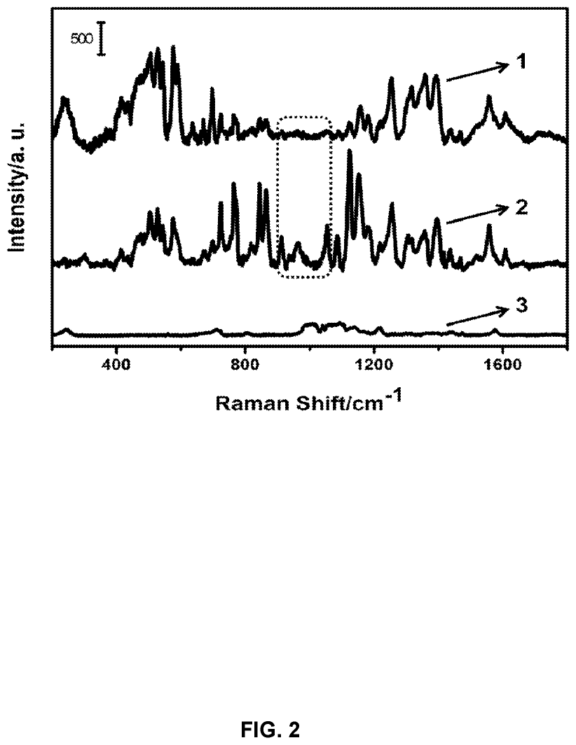

The manner of binding between QR and the bovine insulin was studied by using a Raman spectrometer with an excitation line of a laser model of 532 nm. First, a silver sol was synthesized as a SERS (surface-enhancement raman scattering) substrate. The synthesized silver nanoparticles were respectively added with the bovine insulin (0.01 mM), QR (0.01 mM) and a mixed sample of them, for detection. The detection results are shown in FIG. 2. The results of FIG. 2 showed that between 900-1100 cm-1, there was a signal for QR, while the signal for QR-insulin was disappeared, indicating that insulin bound to a group of QR that had a signal in this wave band.

### Embodiment 3

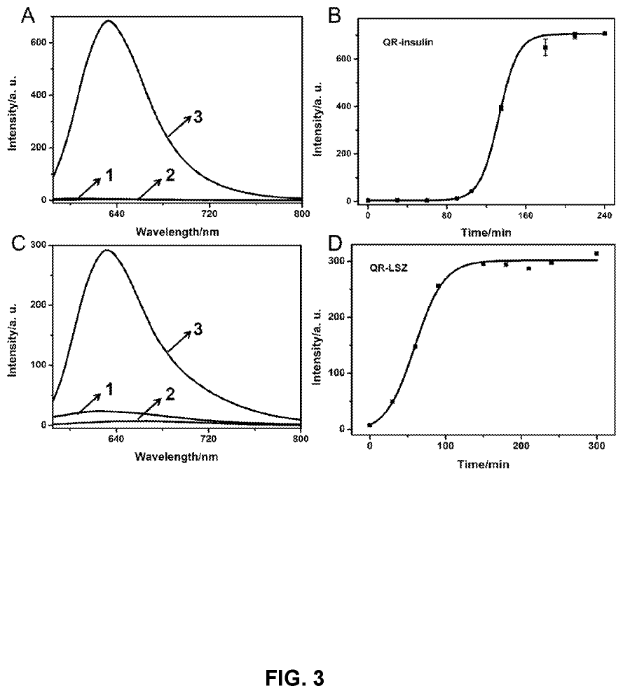

Bovine insulin was added into 1 mL of a NaCl/HCl (25 mM, pH=1.6) solution, where the bovine insulin had a concentration of 0.5 mM, and was incubated at 65° C. for 240 min. QR (0.2 mM), QR-insulin (0.025 mM) and QR-insulin fibril (0.025 mM) were determined for fluorescence intensities respectively. From FIG. 3A, it could be seen that QR had a good identification effect on the bovine insulin fibrils.

As the mechanism of amyloid fibril-related disease was still being explored, and the kinetic process of the amyloid fibrils might provide relevant information for the mechanism, the kinetic process of the formation of the bovine insulin amyloid fibrils was monitored with QR. The bovine insulin was dissolved in 1 mL of a NaCl/HCl (25 mM, pH=1.6) solution, where the bovine insulin had a concentration of 0.5 mM, each 0.05 mL of samples were taken at different time points, then added with 0.05 mL of QR (1 mM) solution, and finally diluted with 0.9 mL of the PBS buffer solution to 1 mL. Three duplicate samples were made for each group of samples, and all of the above samples were determined on a fluorescence spectrometer for fluorescence intensities. From FIG. 3B, it could be found that QR could well detect the dynamic process of bovine insulin fibrillation.

Formulation of a glycine/NaCl buffer solution: the pH value of the glycine/NaCl aqueous solution was adjusted to 2.0 with HCl, where the concentration of sodium chloride was 80 mM and the concentration of glycine was 70 mM.

The lysozyme (0.1 mM) was dissolved in a glycine/NaCl buffer solution for fibrillation at 65° C. under magnetic stirring at a rotation rate of 220 rmp for 180 min. The fluorescence intensities of QR, QR-lysozyme and QR-lysozyme fibrils were determined respectively. From FIG. 3C, it could be seen that the QR had a good identification effect on the lysozyme fibrils.

The kinetic process of the formation of the lysozyme amyloid fibrils was monitored with QR. The lysozyme was dissolved in the glycine/NaCl (pH=2.0) solution, where the lysozyme had a concentration of 0.1 mM, each 0.1 mL of samples were taken at different time points, then added with 0.05 mL of QR (1 mM) solution, and finally diluted with 0.85 mL of a Tris-HCl (200 mM, pH=7.4) buffer solution to 1 mL. Three duplicate samples were made for each group of samples, and all of the above samples were determined on a fluorescence spectrometer for fluorescence intensities. From FIG. 3D, it could be found that QR could well detect the dynamic process of lysozyme fibrillation.

### Embodiment 4

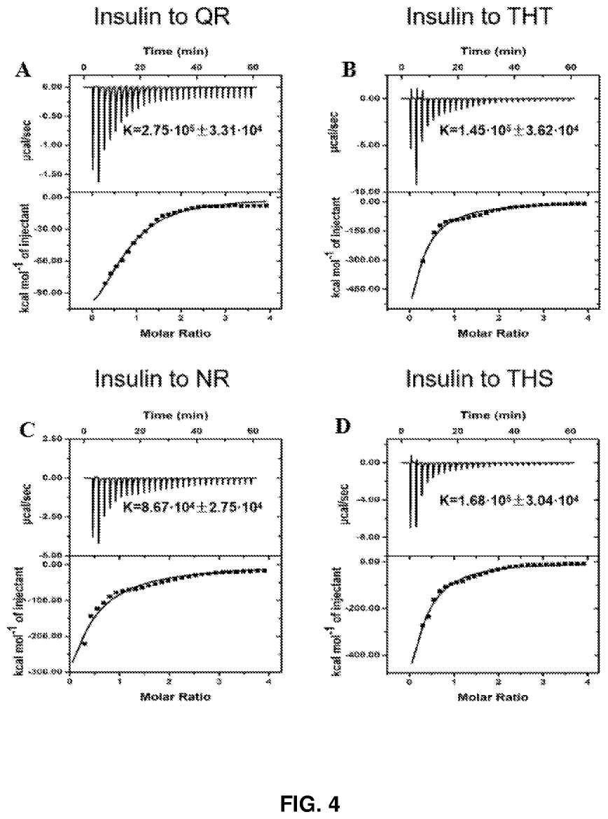

Isothermal titration calorimetry (ITC): the bovine insulin was diluted to a final concentration of 0.25 mM, the QR, ThT, ThS, NR were each diluted to a final concentration of 0.01 mM, and then the aforementioned dyes were titrated with the bovine insulin at 25° C., where the dye was added dropwise into a sample chamber containing a solution of the bovine insulin through an automatically-controlled rotary syringe. At the same time, a blank test was carried out, where dye solutions with equal concentration and equal volume were added dropwise into ultra-pure water, and dilution heat was deducted to obtain a binding constant. The results were shown in FIG. 4, where FIG. 4-A showed titration of QR by the bovine insulin; FIG. 4-B showed titration of ThT by the bovine insulin; FIG. 4-C showed titration of ThS by the bovine insulin; FIG. 4-D showed titration of NR by the bovine insulin. It could be seen from FIG. 4 that QR had the largest binding constant of binding with the bovine insulin.

### Embodiment 5

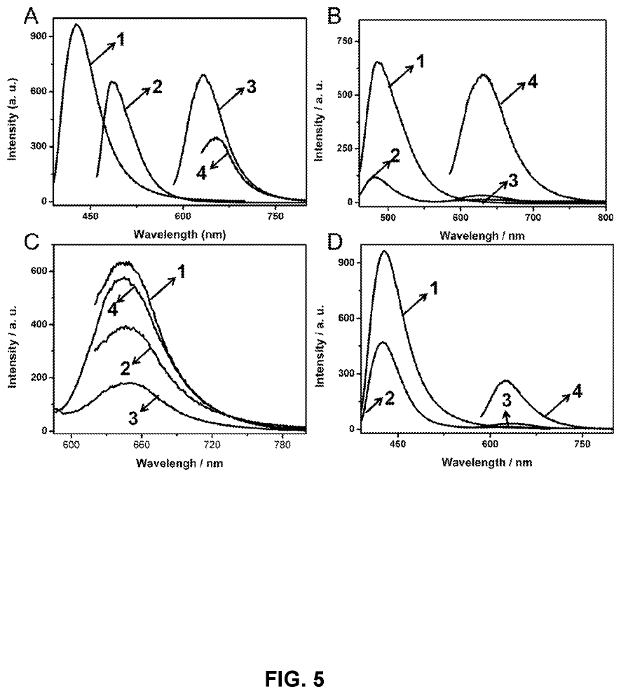

Bovine insulin was added into 1 mL of a NaCl/HCl (25 mM, pH=1.6) solution, where the bovine insulin had a concentration of 0.5 mM, and was incubated at 65° C. for 240 min. 0.05 ml of each of QR, ThT, ThS and NR solutions was taken, then added with 0.05 ml of the bovine insulin fibrils, diluted with 0.9 ml of PBS to 1 ml, and then determined for the fluorescence intensity. The determined results were shown in FIG. 5-A. From FIG. 5-A, it could be seen that each dye could well identify the bovine insulin fibril, but QR had certain advantages of the fluorescence intensity in the red light region.

Since the binding constant of binding between QR and the bovine insulin is the largest, ThT, ThS, and NR were first used to bind to the bovine insulin fibrils for fluorescence determination, then QR was added for a replacement reaction. From FIGS. 5B-5D, it could be seen that the replacement effect was obvious, indicating that QR had greater advantages in a binding force for binding with the bovine insulin fibril.

### Embodiment 6

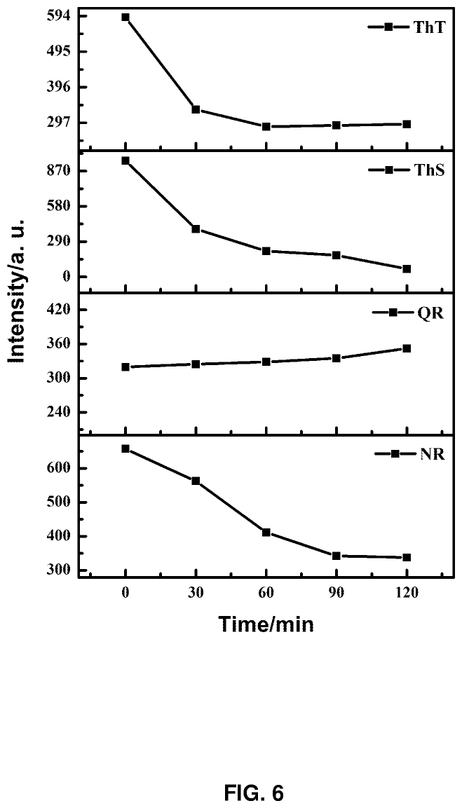

A photobleaching experiment was carried out under ultraviolet light (365 nm). An insulin fibril (0.025 mM) was mixed with QR, ThT, ThS and NR respectively, with the concentrations of all dyes being 0.05 mm. The mixtures were placed under an ultraviolet lamp (365 nm) and measured for fluorescence every 30 minutes. It was found that the fluorescence intensities of ThT+the insulin fibrils, ThS+the insulin fibrils and NR+the insulin fibrils decreased significantly with the increase of the illumination time, while the fluorescence intensity of QR+the insulin fibrils remained basically unchanged (FIG. 6), indicating that QR+the insulin fibrils had good photostability.

### Embodiment 7

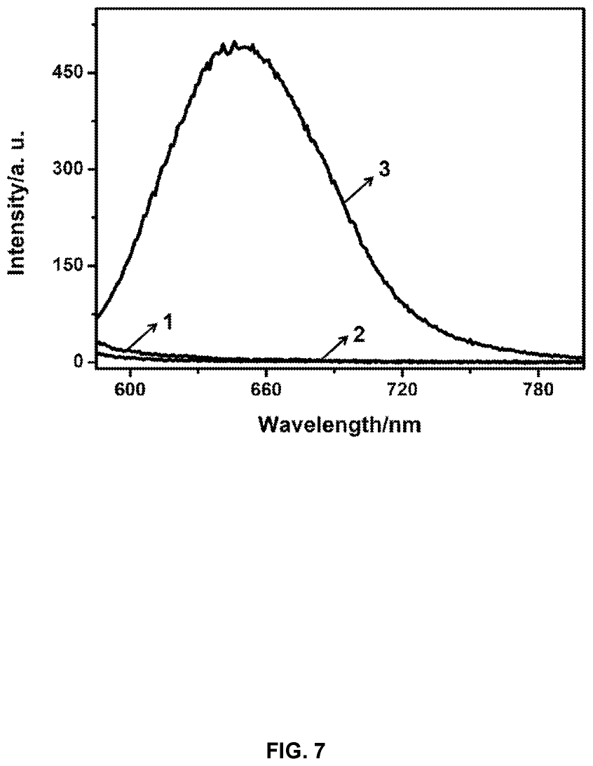

Bovine insulin was added into 1 mL of a NaCl/HCl (25 mM, pH=1.6) solution, where the bovine insulin had a concentration of 0.5 mM, and was incubated at 65° C. for 240 min. The fluorescence intensities of 4-(4-dimethylaminostyryl)quinoline (0.2 mM), 4-(4-dimethylaminostyryl)quinoline-insulin (0.025 mM) and 4-(4-dimethylaminostyryl)quinoline-insulin fibrils (0.025 mM) were determined respectively. The detection results are shown in FIG. 7. As could be seen from FIG. 7, the QR derivative 4-(4-dimethylaminostyryl)quinoline also had a good identification effect on the bovine insulin fibril.

The above description is only preferred embodiments of the present invention. It should be pointed out that, for those of ordinary skill in the art, several improvements and modifications can be made without departing from the principle of the present invention. These improvements and modifications should also be considered as falling into the claimed scope of the present invention.

1. Use of quinaldine red (QR) or a derivative thereof in preparation of a kit for detecting amyloid fibrils, wherein said derivative comprises 4-(4-dimethylaminostyryl)quinoline.

2. The use according to claim 1, wherein the QR or a derivative thereof are used as fluorescent probes in a kit.

3. The use according to claim 1, wherein the amyloid fibrils comprise bovine insulin fibrils, lysozyme fibrils, fibrinogen, and/or Aβ amyloid fibrils.

4. The use according to claim 2, wherein the amyloid fibrils comprise bovine insulin fibrils, lysozyme fibrils, fibrinogen, and/or Aβ amyloid fibrils.

5. A kit for detecting amyloid fibrils comprising a quinaldine red (QR) or a derivative thereof, wherein said derivative comprises 4-(4-dimethylaminostyryl)quinoline.

6. The kit according to claim 5, wherein the QR or a derivative thereof are fluorescent probes that detect the presence of amyloid fibrils.

7. The kit according to claim 5, wherein the amyloid fibrils detectable by the kit comprise bovine insulin fibrils, lysozyme fibrils, fibrinogen, and/or Aβ amyloid fibrils.

8. The kit according to claim 6, wherein the amyloid fibrils detectable by the kit comprise bovine insulin fibrils, lysozyme fibrils, fibrinogen, and/or Aβ amyloid fibrils.

9. A method for detecting amyloid fibrils in a sample comprising contacting the sample with a quinaldine red (QR) or a derivative thereof, wherein said derivative comprises 4-(4-dimethylaminostyryl)quinoline.

10. The method according to claim 9, wherein the presence of amyloid fibrils are detected by a fluorescent signal generated by the QR or a derivative thereof.

11. The method according to claim 9, wherein the amyloid fibrils detected comprise bovine insulin fibrils, lysozyme fibrils, fibrinogen, and/or Aβ amyloid fibrils.

12. The method according to claim 10, wherein the amyloid fibrils detected comprise bovine insulin fibrils, lysozyme fibrils, fibrinogen, and/or Aβ amyloid fibrils.
